SEQUENCE LISTING (6 sequences)

SEQ ID NO: 1 - DNA, 20 bp, Artificial Sequence
  Synthetic Polynucleotide
  misc_feature: ODN1826-3′
  misc_feature (20)..(20): SP18-SP18-X
tccatgacgttcctgacgtt

SEQ ID NO: 2 - DNA, 20 bp, Artificial Sequence
  Synthetic Polynucleotide
  misc_feature: ODN1826-5′ Mod
  misc_feature (1)..(1): X-SP18-SP18
tccatgacgttcctgacgtt

SEQ ID NO: 3 - DNA, 20 bp, Artificial Sequence
  Synthetic Polynucleotide
  misc_feature: GpC-ODN1826-3′ Mod
  misc_feature (20)..(20): SP18-SP18-X
tccatgagcttcctgagctt

SEQ ID NO: 4 - DNA, 20 bp, Artificial Sequence
  Synthetic Polynucleotide
  misc_feature: GpC-ODN1826-5′-Mod
  misc_feature (1)..(1): X-SP18-SP18
tccatgagcttcctgagctt

SEQ ID NO: 5 - DNA, 20 bp, Artificial Sequence
  Synthetic Polynucleotide
  misc_feature: Complement ODN1826-3′ Mod
  misc_feature (20)..(20): SP18-SH
aacgtcaggaacgtcatgga

SEQ ID NO: 6 - DNA, 20 bp, Artificial Sequence
  Synthetic Polynucleotide
  misc_feature: Complement ODN 1826-5′ Mod
  misc_feature (1)..(1): SH-SP18
aacgtcaggaacgtcatgga

(19) United States
(12) Patent Application Publication
(10) Pub. No.: US 2022/0010302 A1
(43) Pub. Date: Jan. 13, 2022

(54) ADDRESSING NANOMEDICINE COMPLEXITY THROUGH NOVEL HIGH-THROUGHPUT SCREENING AND MACHINE LEARNING

(71) Applicant: NORTHWESTERN UNIVERSITY, Evanston, IL (US)

(72) Inventors: Chad A. Mirkin, Wilmette, IL (US); Gokay Yamankurt, Chicago, IL (US); Milan Mrksich, Hinsdale, IL (US); Eric J. Berns, Park Ridge, IL (US); Neda Bagheri, Chicago, IL (US); Albert Xue, Chicago, IL (US); Andrew Lee, Evanston, IL (US)

(21) Appl. No.: 17/046,482

(22) PCT Filed: Apr. 12, 2019
(86) PCT No.: PCT/US2019/027229

§ 371 Date: Oct. 9, 2020

Related U.S. Application Data
(60) Provisional application No. 62/657,441, filed on Apr. 13, 2018.

(51) Int. Cl. C12N 15/10

(52) U.S. Cl. CPC C12N 15/1086

(57) ABSTRACT

The present disclosure provides methods for the rapid synthesis of large libraries of spherical nucleid acid (SNA) nanoparticles, their screening for activity, and a machine learning algorithm to analyze the data.

### CROSS REFERENCE TO RELATED APPLICATIONS

This application claims the priority benefit under 35 U.S.C. § 119(e) of U.S. Provisional Patent Application No. 62/657,441, filed Apr. 13, 2018, the disclosure of which is incorporated herein by reference in its entirety.

### STATEMENT OF GOVERNMENT INTEREST

This invention was made with government support under U54CA199091-01 and U54CA151880-01, each awarded by the National Institutes of Health. The government has certain rights in the invention.

### FIELD OF THE INVENTION

The present disclosure provides methods for the rapid synthesis of large libraries of spherical nucleic acid (SNA) nanoparticles, their screening for activity, and a machine learning algorithm to analyze the data.

### BACKGROUND

Nanotechnology is beginning to play a major role in developing new therapeutic modalities. Currently, over 100 drugs based upon nanomaterials are currently in clinical trials or approved for therapeutic use¹. These structures are promising because of their multifunctionality, which directly relates to their relatively large size and often complex architectures when compared with conventional small molecules or biologics. However, due to this complexity, little attention has been paid to how structural changes inform biological activity. Consider, for example, spherical nucleic acids (SNAs), structures made by arranging short sequences of DNA or RNA around a nanoparticle core. (FIG. 1a)^{2,3}. SNAs exhibit properties that are completely different from the short linear oligonucleotides that comprise them, including the ability to actively cross mammalian cell membranes without the need for transfection reagents, a resistance to nuclease degradation, and the ability to carry large and complex cargo (such as oligonucleotides and peptides) into many cell types.⁴⁻⁷

Because of these properties, SNAs have shown promise in cancer immunotherapy, where structures with dual functionality can be rapidly prepared from lipids, oligonucleotide adjuvants, and peptide antigens. When delivered to antigen presenting cells (APCs), SNAs activate the immune system and, in a lymphoma model, have shown superior activity compared to the same free antigen and linear oligonucleotides⁵. However, the modularity of an SNA allows for a large number of possible designs, and the best nanoparticle architectures for maximizing potency and efficacy are unclear.

### SUMMARY OF THE INVENTION

The present disclosure provides a high throughput method for making different forms of SNAs that are qualitatively similar but structurally distinct, and a mass spectrometry-based screening protocol that allows one to rapidly determine activity for enzyme activation. Collectively, these insights are useful in designing SNA-based therapeutics. Further, in light of the fact that the methodology can be extended to other nanotherapeutics, the present disclosure provides a new way of designing and optimizing nanomedicines for a wide variety of uses.

Accordingly, in some aspects the disclosure provides a method of screening activity of a library of oligonucleotide-functionalized spherical nucleic acids (SNAs) comprising: (a) individually contacting each SNA of the library with a cell, wherein upon contact with the SNA, the cell modulates expression of an enzyme and the amount of enzyme expressed is in proportion to the activity of the SNA; (b) contacting the enzyme expressed in step (a) with a substrate under conditions to transform the substrate to a product, wherein the product has a mass different from the substrate; (c) immobilizing the product and the substrate on a self-assembled monolayer (SAM) on a surface; (d) subjecting the immobilized substrate and product to mass spectrometry to produce a mass spectrum having a product signal and a substrate signal; and (e) correlating the product signal intensity to the substrate signal intensity to determine the extent of product formation and thereby assay the activity of each SNA.

In some embodiments, at least one SNA in the library further comprises an antigen. In further embodiments, the SNAs of the library differ in at least one structural parameter, and the structural parameter is a SNA core property, an antigen property, an oligonucleotide property, or a combination thereof. In still further embodiments, the SNA core property is core diameter, core composition, or a combination thereof. In some embodiments, the core diameter is from about 30 nanometers (nm) to about 150 nm in mean diameter. In further embodiments, the core composition is 1,2-dioleoyl-sn-glycero-3-phosphocholine (DOPC), 1,2-di-(9Z-octadecenoyl)-sn-glycero-3-phosphoethanolamine (DOPE), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-dihexadecanoyl-sn-glycero-3-phosphoethanolamine (DPPE), or a combination thereof. In still further embodiments, the antigen property is antigen composition, antigen location, antigen density, or a combination thereof.

In some embodiments, the antigen composition comprises human papillomavirus (HPV) E7 protein or ovalbumin (OVA). In further embodiments, the antigen location is encapsulated within the core or associated with the outer surface of the core. In some embodiments, the antigen is associated with the oligonucleotide that is functionalized on the outer surface of the core.

In some embodiments, the at least two SNAs differ from each other in that one SNA comprises 2×, 3×, 4×, 5×, 6×, 7×, 8×, 9×, or 10× of encapsulated antigen relative to a second SNA. In some embodiments, the at least two SNAs differ from each other in that one SNA has 0%, 10%, 20%, 30%, 40%, 50%, 60%, 70%, 80%, 90%, or 100% of the oligonucleotide on the outer surface of the core associated with antigen relative to a second SNA.

In some embodiments, the oligonucleotide property is oligonucleotide sequence, oligonucleotide conjugation chemistry, oligonucleotide conjugation terminus, oligonucleotide backbone, oligonucleotide density, complement density, or a combination thereof. In some embodiments, the at least two SNAs differ from each other in that one SNA comprises a density of oligonucleotide on its outer surface that is 2×, 3×, 4×, 5×, 6×, 7×, 8×, 9×, or 10× that of a density of oligonucleotide on the outer surface of a second SNA. In further embodiments, the at least two SNAs differ from each other in that one SNA has 0%, 10%, 20%, 30%, 40%, 50%, 60%, 70%, 80%, 90%, or 100% of the oligonucleotide on the outer surface of the core associated with a complementary oligonucleotide relative to a second SNA.

In further embodiments, the oligonucleotide sequence activates a Toll-like receptor (TLR). In some embodiments, the TLR is TLR-9. In still further embodiments, the oligonucleotide sequence comprises a CpG motif.

In some embodiments, the oligonucleotide conjugation chemistry is a cholesterol-modified oligonucleotide or a 1,2-di-(9Z-octadecenoyl)-sn-glycero-3-phosphoethanolamine (DOPE)-modified oligonucleotide. In further embodiments, the oligonucleotide conjugation terminus is a 5′ terminus of the oligonucleotide or a 3′ terminus of the oligonucleotide.

In some embodiments, the oligonucleotide backbone is a phosphodiester (PO) backbone or phosphorothioate (PS) backbone.

In some embodiments, each SNA in the library of oligonucleotide-functionalized SNAs is in a separate well of a multiwell plate. In further embodiments, the SAM comprises an immobilizing moiety that interacts with and immobilizes the substrate and the product. In still further embodiments, the immobilizing moiety comprises a maleimide, a thiol, an alkyne, an azide, an amine, or a carboxyl group. In some embodiments, (i) the immobilizing moiety comprises a maleimide and the substrate and the product each comprise an alkane thiol; (ii) the immobilizing moiety comprises an alkane thiol and the substrate and the product each comprise a maleimide; (iii) the immobilizing moiety comprises an alkyne and the substrate and the product each comprise an azide; (iv) the immobilizing moiety comprises an azide and the substrate and the product each comprise an alkyne; (v) the immobilizing moiety comprises an amine and the substrate and the product each comprise a carboxyl group; or (vi) the immobilizing moiety comprises a carboxyl group and the substrate and the product each comprise an amine, so as to form a chemical bond between the immobilizing moiety and the substrate.

In some embodiments, the enzyme is a deacetylase, acetyltransferase, esterase, phosphorylase/kinase, phosphatase, protease, methylase, demethylase, or a DNA or RNA modifying enzyme. In further embodiments, the phosphatase is secreted embryonic alkaline phosphatase (SEAP). In further embodiments, the deacetylase is KDAC8. In still further embodiments, the esterase is cutinase or acetylcholine esterase. In some embodiments, the protease is TEV. In some embodiments, the substrate comprises an acylated peptide and the product comprises a deacylated peptide. In further embodiments, the substrate comprises a deacylated peptide and the product comprises an acylated peptide In some embodiments, the substrate comprises a phosphorylated peptide and the product comprises a dephosphorylated peptide. In further embodiments, the substrate comprises a dephosphorylated peptide and the product comprises a phosphorylated peptide. In some embodiments, the substrate comprises a methylated peptide and the product comprises a demethylated peptide. In some embodiments, the substrate comprises a demethylated peptide and the product comprises a methylated peptide.

### BRIEF DESCRIPTION OF THE FIGURES

FIG. 1 shows: a, Components of immunostimulatory spherical nucleic acids (SNAs). b, The parameters investigated for each of the SNA design properties, organized by core, antigen, and oligonucleotide property categories. c, The total design space investigated in this study, divided into three subsets.

FIG. 2 shows: a, The assay used to evaluate the structure-activity relationships between SNA properties and TLR9 activation of APCs: Libraries of SNAs are incubated with RAW-Blue macrophages, engineered to secrete SEAP (a phosphatase) into the media, in 384-well plates. After approximately 16 hours, media is transferred, processed, and mixed with a phosphorylated substrate. The solution is transferred to SAMDI plates with 1536-spot arrays of monolayers presenting maleimides to selectively capture the substrate and product by a maleimide-thiol reaction. b, An example SAMDI spectrum showing the immobilized substrate and product. Performing MALDI-MS on the self-assembled monolayers (i.e., SAMDI) results in mass spectra containing quantitative information on the relative amounts of substrate and product (i.e., extent of dephosphorylation). c, An example standard curve used to convert the SAMDI spectral data for the library into SEAP concentration.

FIG. 3 shows: a, The SEAP concentrations observed for all active-sequence SNAs in the encapsulated OVA subset (all data in this figure are from this subset), compared to the PO- and PS versions of linear oligonucleotides with the same active sequence. b, Comparison of SNAs with the active and control sequences, grouped into the SNAs with cholesterol-conjugated oligonucleotides and c, DOPE-conjugated oligonucleotides. d, A dimension stacking plot of the active-sequence SNAs, showing the SEAP concentration for each combination of design properties. Larger and darker circles indicate greater SEAP concentration. e and f, Comparison of 5′ and 3′ conjugation termini of SNAs with active sequence, grouped by conjugation chemistry. g and h, Comparison of PO and PS backbones of SNAs with active sequence, grouped by conjugation chemistry.

FIG. 4 depicts a dimension-stacking plot of the active-sequence SNAs in the encapsulated E7 subset, showing the SEAP concentration for each combination of design properties. Larger and darker circles indicate greater SEAP concentration.

FIG. 5 shows: a, The difference in SEAP concentration between each SNA with the highest peptide concentration (10×) and the corresponding SNA without any peptide (Ox), for the two encapsulated peptide subsets. b and c, The average difference in SEAP concentration between SNAs with 10× and Ox E7 peptide concentration, grouped by core diameter, the combination of conjugation chemistry and lipid composition, for the E7 and OVA subsets. Chol, C/E indicates cholesterol conjugation and 80% DOPC/20% DOPE lipid composition. Chol, C indicates cholesterol conjugation and 100% DOPE lipid composition. DOPE, C indicates DOPE conjugation and 100% DOPE lipid composition (Chol, C/E and Chol, C: n=16, DOPE, C: n=24; *: P<0.05, ***: P<0.001).

FIG. 6 shows: a, The SEAP concentrations for all of the active-sequence SNAs in the surface-presented OVA subset. b, The mean SEAP concentration of PS-backbone, active-sequence SNAs, grouped by the combinations of complement density and surface antigen density. (n=12) c, The mean SEAP concentration of SNAs with PS-backbone, 0% peptide and active-sequence, as a function of complement density, at 10 nM oligonucleotide concentration. (n=12) d, The mean SEAP concentration of SNAs with PO-backbone and active-sequence, as a function of complement density, at 1000 nM oligonucleotide concentration. (0%: n=36, 50%: n=24, 0%: n=12; **: P<0.01, ***: P<0.001).

FIG. 7 shows: a, The Q² of the highest performing SNA property combinations are shown across different numbers of properties for encapsulated OVA and b, surface-presented OVA subsets. For the encapsulated OVA subset and xgboost model, the active sequence and 100 nM subset is shown (Δ) in addition to both active/inactive sequences with all concentrations (o). c, Xgboost Q² performance when selecting and training on a random SNA subsample and testing predictions on the unselected SNAs or d, cross-validating within the selected subsample. All plots have 90% confidence intervals.

FIG. 8 shows: a, Highest Q² scoring property combinations are shown across different number of properties for encapsulated OVA subset, b surface-presenting OVA subset, and c encapsulated OVA subset with active sequence and 100 nM. Bubble areas correspond to Q² values from FIG. 7. Orange and purple properties denote exclusive and shared properties between the two subsets, respectively.

FIG. 9 depicts the non-observable external Q² (predicting immune activity of non-synthesized SNAs from a synthesized subsample) is plotted against the observable internal Q² (cross-validating within the synthesized subsample) for all three subsets. The median line and 90%, 50% and 20% confidence intervals are shown.

### DETAILED DESCRIPTION

A tiny fraction of the nanomedicine design space has been explored, in large part, due to the complexities of such structures and the lack of high-throughput methods to make and analyze them. To address this challenge, the present disclosure provides methods for testing spherical nucleic acids (SNAs), which have over ten different parameters that can be systematically and independently changed to optimize performance. In some embodiments, the performance of the SNAs is optimized in the context of immune cell activation.

By focusing on reasonable parameter ranges, thousands of therapeutic candidate structures have been identified herein that are qualitatively similar but could have significant differences in activity, thereby creating both a synthesis and an analysis challenge. To overcome this daunting task, a high-throughput method for making such structures at picomole (pmol) scale in 384-well format, and a self-assembled monolayer matrix desorption ionization (SAMDI) mass spectrometry assay to rapidly measure innate activation of an enzyme by quantitatively determining enzyme activity. Traditionally, cell-based optical assays are used for measuring such activity, but they are susceptible to optical artifacts due to the absorption and scattering of light associated with the nanostructures that define the SNAs. Through the methods described herein, structure-activity relationships between SNAs and enzyme activation (e.g., immune activation) are identified, which provide new design rules for SNA-based therapeutics (e.g., cancer vaccine candidates). Finally, machine learning was utilized to quantitatively model the enzyme activation of SNAs, and then applied to identify the minimum number of SNAs needed to capture optimum structure-activity relationships for a given library. By doing so, one can reduce the number of nanoparticles to be tested by an order of magnitude, and still get the same information from screening the entire library. These insights and techniques can be generalized to include many other types of nanomedicines and provide a next generation screening tool for therapeutic development.

### The Modular Design of Spherical Nucleic Acids

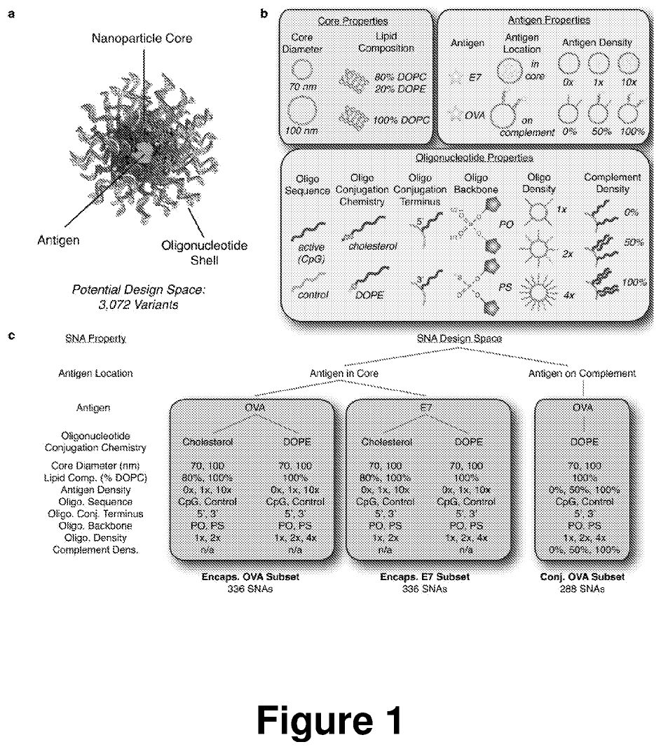

In some aspects and embodiments of the disclosure, methods are provided for creating immunostimulatory SNAs. Immunostimulatory SNAs consist of three components: the nanoparticle core, the oligonucleotide shell, and the peptide antigen, all of which can be arranged in a variety of configurations⁵. To establish an appropriate library for high-throughput assessment, focus was placed on eleven properties across these components—core diameter, lipid composition, antigen, antigen location (in core or on complement), antigen density, oligonucleotide sequence, oligonucleotide conjugation chemistry, oligonucleotide conjugation terminus (e.g., 3′ or 5′), oligonucleotide backbone, oligonucleotide density, and complement density (FIG. 1b). In some embodiments, 1,2-dioleoyl-sn-glycero-3-phosphocholine (DOPC) and 1,2-dioleoyl-sn-glycero-3-phosphoethanolamine (DOPE) is used to form liposomes that are biocompatible, easy to synthesize, and capable of encapsulating the antigen⁸. Various liposomal diameters are contemplated by the disclosure. In some embodiments, liposome core sizes with average diameters of approximately 70 and approximately 100 nm are utilized and are produced from DOPC or a mixture of 80% DOPC and 20% DOPE, respectively. The size of the SNA can influence its rate of cellular uptake, and inclusion of DOPE in the liposomes is believed to affect the peptide release rate and their endosomal escape, which is important for peptide processing^{9,10}. Thus, in various embodiments, an SNA created by a method of the disclosure is less than or equal to about 50 nanometers. In some embodiments, a plurality of SNAs is produced and the SNAs in the plurality have a mean diameter of less than or equal to about 50 nanometers (e.g., about 5 nanometers to about 50 nanometers, or about 5 nanometers to about 40 nanometers, or about 5 nanometers to about 30 nanometers, or about 5 nanometers to about 20 nanometers, or about 10 nanometers to about 50 nanometers, or about 10 nanometers to about 40 nanometers, or about 10 nanometers to about 30 nanometers, or about 10 nanometers to about 20 nanometers). In further embodiments, the SNAs in the plurality have a mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers.

The oligonucleotide shell serves two roles. It facilitates cellular uptake and serves as the adjuvant, which activates the innate immune system in a sequence-specific and orientation-dependent manner⁵. In embodiments wherein the SNA is an immunostimulatory SNA, a CpG DNA oligonucleotide (ODN1826), known to activate mouse Toll-like receptor 9 (TLR9), may be utilized. In further embodiments, an inactive control SNA where the CpG motif is inverted to GpC^{11,12} is also produced. TLR9 is an endosomal protein that recognizes unmethylated CpG oligonucleotides associated with bacteria and viruses¹³. To explore the importance of backbone composition, linear oligonucleotides with phosphodiester (PO) or phosphorothioate (PS) backbones were synthesized because PS oligonucleotides are known to induce higher immune activation, but SNAs yield PO structures with activities comparable to PS, structures^{5,14}. Thus, in various embodiments, SNAs are contemplated that comprise oligonucleotides having PO or PS backbones. Oligonucleotide-nanoparticle conjugation was studied by investigating structures conjugated with cholesterol or DOPE, which insert into the liposomes and can be chemically attached to the 3′- or 5′ ends of the oligonucleotides. Finally, since oligonucleotide density is known to influence cellular uptake and protein binding of SNAs, various oligonucleotide surface densities are contemplated. These properties and variations thereof are discussed further herein below.

The antigen is not particularly limiting and can be any antigen of interest for a protein of interest. In some embodiments, the peptide antigen is the OVA257-264 peptide from ovalbumin, a well-studied model antigen. In further embodiments, the peptide antigen is Glycoprotein 100 (Gp100), human papillomavirus antigens (including, without limitation, E6 and E7), prostate-specific antigen (PSA), prostate-specific membrane antigen (PSMA), or transmembrane AMPA receptor Regulatory Proteins (TARP). Since peptide properties can vary dramatically as a function of amino acid composition, a peptide antigen from the E7 protein of the human papillomavirus¹⁷ is also described and tested herein. To study how the release rate of the antigen influences NF-κB activation, variations in which the antigen is encapsulated within the SNA architecture or hybridized to the oligonucleotide shell through a complementary oligonucleotide are contemplated as well as tested herein. In further embodiments, addition of a complementary oligonucleotide is contemplated for its effects on TLR (e.g., TLR9) stimulation.

### High-Throughput Screening of SNA Libraries

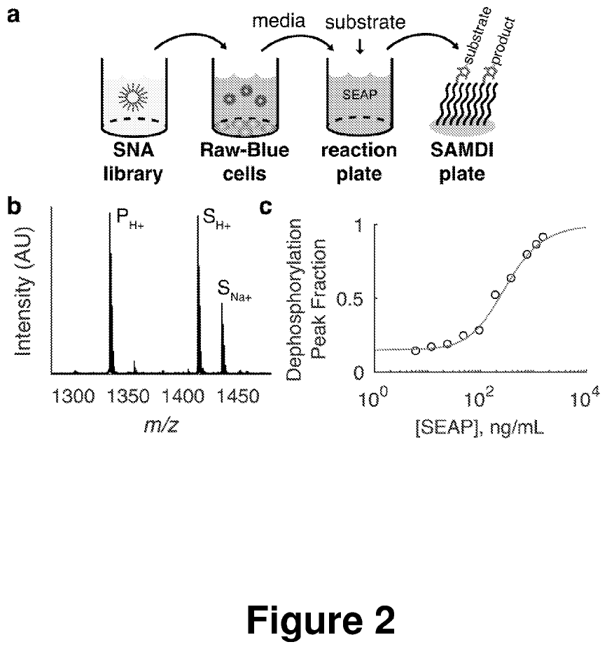

To enable screening SNA libraries, a high-throughput assay for rapid and quantitative measurement of cellular responses to SNAs was developed (FIG. 2a). In a specific, non-limiting example, RAW-Blue macrophages are cultured in 384-well plates and are treated with SNAs at multiple oligonucleotide concentrations. In some embodiments, the cells are treated with SNAs at about four different oligonucleotide concentrations. In further embodiments, the cells are treated with SNAs at concentrations that are between 1 nanomolar (nM) and 1 micromolar (μM). RAW-Blue cells are engineered to secrete embryonic alkaline phosphatase (SEAP) upon activation of NF-κB, a major transcription factor that regulates the immune response. Next, culture media is collected and the concentration of secreted enzyme (e.g., SEAP) is determined using SAMDI (Self-Assembled monolayers and MALDI) mass spectrometry, a platform well-suited for high-throughput, quantitative analysis of enzymatic activityl⁸⁻²¹. SAMDI uses monolayers presenting a selective capture chemistry against a background of non-binding tri(ethylene glycol) to isolate substrates and products from a complex mixture^{21,22}. Subsequently, MALDI-MS of the monolayers measures the relative amounts of substrate and product, which is used to determine the enzyme concentration (FIGS. 2b and c).

Self-Assembled Monolayer (SAM) Surfaces. The present disclosure contemplates the use of self-assembled monolayers as surfaces for assay applications (Mrksich et al., Annu Rev Biophys Biomol Struct 25: 55-78 (1996); Hodneland et al., Langmuir 13: 6001-6003 (1997); Houseman et al., FASEB J 11: A1095-A1095 (1997); Mrksich, Curr Opin Colloid In 2: 83-88 (1997); Mrksich et al., Acs Sym Ser 680: 361-373 (1997); Houseman et al., Mol Biol Cell 9: 430a-430a (1998); Mrksich, Cell Mol Life Sci 54: 653-662 (1998); Houseman et al., Angew Chem Int Ed 38: 782-785 (1999); Li et al., Langmuir 15: 4957-4959 (1999); Yousaf et al., J Am Chem Soc 121: 4286-4287 (1999); Houseman et al., Mol Biol Cell 11: 45a-45a (2000); Luk et al., Langmuir 16: 9604-9608. (2000); Mrksich, Chem Soc Rev 29: 267-273 (2000); Yousaf et al., Angew Chem Int Ed Engl 39: 1943-1946 (2000); Yousaf et al., Biochemistry 39: 1580-1580 (2000); Houseman et al., Biomaterials 22: 943-955 (2001); Kato et al., Biochemistry 40: 8608-8608 (2001); Yeo et al., Chembiochem 2: 590-593 (2001); Yousaf et al., Proc Natl Acad Sci USA 98: 5992-5996. (2001); Yousaf et al., Angew Chem Int Ed Engl 40: 1093-1096 (2001); Hodneland et al., Proc Natl Acad Sci USA 99: 5048-5052 (2002); Houseman et al., Nat Biotechnol 20: 270-274 (2002); Houseman et al., Top Curr Chem 218: 1-44 (2002); Houseman et al., Trends Biotechnol 20: 279-281 (2002); Houseman et al., Chem Biol 9: 443-454 (2002); Kwon et al., J Am Chem Soc 124: 806-812 (2002); Lee et al., Science 295: 1702-1705 (2002); Mrksich, Curr Opin Chem Biol 6: 794-797 (2002); Houseman et al., Langmuir 19: 1522-1531 (2003); Luk et al., Biochemistry 42: 8647-8647 (2003); Yeo et al., Angew Chem Int Ed Engl 42: 3121-3124 (2003); Dillmore et al., Langmuir 20: 7223-7231 (2004); Feng et al., Biochemistry 43: 15811-15821 (2004); Kato et al., J Am Chem Soc 126: 6504-6505 (2004); Min et al., Curr Opin Chem Biol 8: 554-558 (2004); Murphy et al., Langmuir 20: 1026-1030 (2004); Yeo et al., Adv Mater 16: 1352-1356 (2004); Yonzon et al., J Am Chem Soc 126: 12669-12676 (2004); Mrksich, MRS Bull 30: 180-184 (2005); James et al., Cell Motil Cytoskeleton 65: 841-852 (2008)). Previous work utilized a monolayer that presented a peptide against a background of tri(ethylene glycol) groups (Houseman et al., Nat Biotechnol 20: 270-274 (2002)). The peptide was a substrate for Src kinase and the glycol groups prevented non-specific adsorption of protein to the monolayer. Treatment of the monolayer with enzyme and ATP resulted in phosphorylation of the peptide, which was detected by measuring radioactivity from a ³²P label or by using an anti-phosphotyrosine antibody with detection by fluorescence scanning or surface plasmon resonance spectroscopy. This example showed that the use of monolayers gave solid-phase assay with exceptional performance. It further indicated that blocking procedures were unnecessary; the signal was 80-fold above background; and that enzyme constants and inhibitor dissociation constants could be measured quantitatively. The monolayers offer the benefits that immobilized ligands are presented in a homogeneous environment and the density of the immobilized ligands can be controlled and made uniform across the entire array (Gawalt et al., J Am Chem Soc 126: 15613-7 (2004)). The monolayers are also compatible with a range of immobilization chemistries (Montavon et al., Nat Chem 4: 45-51 (2012); Ban et al., Nat Chem Biol 8: 769-773 (2012); Li et al., Langmuir 23, 11826-11835 (2007)). In these respects, the monolayers are more effective as substrates in assay applications than is the nitrocellulose material, or even the common use of glass. A significant additional benefit of the monolayer substrates is that they can be analyzed by matrix-assisted laser desorption-ionization mass spectrometry (i.e., SAMDI mass spectrometry) and therefore provide a route to label-free assays of biochemical activities (Su et al., Langmuir 19: 4867-4870 (2003)).

### SAMDI Mass Spectrometry

SAMDI mass spectrometry (MS) can be used to detect the mass of a substrate or product. The monolayer is reacted with the substrate and product formed by the enzyme to form a covalent bond with each of the substrate and product on the monolayer. Then, the monolayer is subjected to mass spectrometry, and due to the mass difference between the substrate and product, the MS analysis can assess how much substrate and product are present based upon a single MS analysis. SAMDI can be performed in high throughput using plates having a number of distinct reaction zones (e.g., 1536 or 384) offering a throughput of about 50,000 assays per day, and is quantitative with Z-factors greater than 0.8. The assay can also be used to screen the activity of the antigens of interest in the assays described herein to identify inhibitors or activators of enzymes of interest.

In SAMDI, the monolayer is irradiated with a laser, which results in desorption of the products and substrates through dissociation of a thiolate-gold bond, but with little fragmentation of these molecules. Hence, the resulting spectra are straightforward to interpret. Assays using this SAMDI technique can be used on a range of enzyme activities, and are quantitative, compatible with complex lysates, and adaptable to high throughput formats (Ban et al., Nat Chem Biol 8: 769-773 (2012); Li et al., Langmuir 23: 11826-11835 (2007); Su et al., Langmuir 19: 4867-4870 (2003); Su et al., Angew Chem Int Ed Eng. 41: 4715-4718 (2002); Min et al., Angewandte Chemie 43: 5973-5977 (2004); Min et al., Anal Chem 76: 3923-3929 (2004); Yeo et al., Angew Chem Int Ed Engl 44: 5480-5483 (2005); Marin et al., Angew Chem Int Ed Engl 46: 8796-8798 (2007); Patrie et al., Anal Chem 79: 5878-5887 (2007); Ban et al., Angew Chem Int Ed Eng 47: 3396-3399 (2008); Gurard-Levin et al., Annu Rev Anal Chem (Palo Alto Calif.) 1: 767-800 (2008); Gurard-Levin et al., Biochemistry 47: 6242-6250 (2008); Mrksich, ACS Nano 2: 7-18 (2008); Tsubery et al., Langmuir 24: 5433-5438 (2008); Gurard-Levin et al., Chembiochem 10: 2159-2161 (2009); Liao et al., Chemistry 15, 12303-12309 (2009); Gurard-Levin et al., ACS Chem Biol 5: 863-873 (2010); Kim et al., Nucleic Acids Res 38: e2 (2010); Cai et al., Carbohydr Res 346: 1576-1580 (2011); Gurard-Levin et al., ACS Comb Sci 13: 347-350 (2011); Liao et al., Angew Chem Int Ed Engl 50: 706-708 (2011); Prats-Alfonso et al., Small 8: 2106-2115 (2012); Li et al., Langmuir 29: 294-298 (2013)).

In general, the disclosure provides methods of screening activity of a library of oligonucleotide-functionalized spherical nucleic acids (SNAs) comprising: (a) individually contacting each SNA of the library with a cell, wherein upon contact with the SNA, the cell modulates expression of an enzyme and the amount of enzyme expressed is in proportion to the activity of the SNA; (b) contacting the enzyme expressed in step (a) with a substrate under conditions to transform the substrate to a product, wherein the product has a mass different from the substrate; (c) immobilizing the product and the substrate on a self-assembled monolayer (SAM) on a surface; (d) subjecting the immobilized substrate and product to mass spectrometry to produce a mass spectrum having a product signal and a substrate signal; and (e) correlating the product signal intensity to the substrate signal intensity to determine the extent of product formation and thereby assay the activity of each SNA.

The methods described herein offer several advantages. First, the technology to synthesize SNA libraries did not exist prior to the instant disclosure. The methods disclosed herein enable the use of large numbers of SNAs to understand their behavior and screen libraries to find the best SNAs for a given purpose. Second, current technologies based on optical technologies to detect enzyme activation suffer from artifacts in nanoparticle testing due to the ability of nanoparticles to interact with light. Although it is possible to correct for these artifacts, they introduce many steps that make the screen infeasible. The methods of the disclosure use mass spectrometry to detect enzyme activation (e.g., immune activation) and is not susceptible to these types of artifacts. Finally, the assay can measure activities from enzymes, such as phosphatases, which are impractical to measure in high-throughput from cell lysates using other assay technologies.

Cell-based screening is an increasingly popular tool used in drug discovery. This technology opens up the potential of conducting cell-based screens that use enzyme activity measurements as the readout. This is of significant value because cell-based screens provide more physiologically relevant information about the activity of compounds, potentially leading to better lead compounds in drug discovery efforts.

Surface. The surface can be any material capable of forming a monolayer, e.g., a monolayer of alkanethiols. Particularly, the substrate may be a metal, such as Au, Ag, Pd, Pt, Cu, Zn, Fe, In, Si, Fe₂O₃, SiO₂ or ITO (indium tin oxide) glass. In various embodiments, the disclosure contemplates that a surface useful in the methods described herein comprises Au, Ag, or Cu. In some cases, the surface comprises Au.

In some embodiments, the disclosure contemplates that the substrate and the product comprise a moiety capable of reacting with the SAM so as to be immobilized on the SAM for SAMDI analysis, e.g., a maleimide, a thiol, an alkyne, an azide, an amine, or a carboxyl group. This immobilizing moiety can react with the SAM to form a covalent bond, for example the substrate and product or SAM comprises a maleimide and the other comprises an alkane thiol; the substrate and product or SAM comprises an alkyne and the other comprises an azide; the substrate and product or SAM comprises an amine and the other comprises a carboxyl group.

Intracellular enzyme. The disclosure generally provides methods of assaying activity of an intracellular enzyme. It is contemplated that, in some aspects, the enzyme to be assayed is secreted from the cell. Any enzyme is contemplated for use according to the methods provided herein, including but not limited to a deacetylase, acetyltransferase, esterase, phosphorylase/kinase, phosphatase, protease, methylase, demethylase, or a DNA or RNA modifying enzyme. In some embodiments, the enzyme is a secreted enzyme. Thus, in some embodiments the enzyme is secreted embryonic alkaline phosphatase.

High Throughput Formats for SAMDI. SAMDI for use in the disclosed methods uses a high throughput format based on standard 384 and 1536 microtiter plate formats. This format uses a stainless steel plate in the size of a microtiter plate and having an array of gold-coated islands modified with a monolayer presenting a reactive group that can react with an immobilizing moiety on the substrate and product to form a covalent bond (e.g., maleimide groups) against a background of tri(ethylene glycol) groups. Substrates and products are then immobilized to each of the islands; in various embodiments, in a high throughput screen each island has the same substrate and product produced in response to the antigen of interest for a single SNA, to identify active SNAs for an enzyme. Standard robotic liquid handling equipment can be used to prepare arrays of reactions and to transfer those reaction mixtures to the array plates. The treated plates are incubated (e.g., between 30-60 minutes), washed, and a solution of matrix is applied to the surface. The plate is then loaded into a MALDI-ToF instrument, and each spot is analyzed in an automated fashion in approximately 30 minutes. Resulting data is analyzed using custom written software that can compare the location and magnitude of the peaks in the SAMDI spectra to a reference mass file unique to each set of peptides to look for specific reaction profiles based on characteristic mass shifts (i.e., −42 for deacetylation, +80 for phosphorylation, +14 for methylation). The software presents the data in a manner that can be further analyzed with standard commercial packages (such as Excel) to identify hits in a high throughput screen, or to generate heatmaps of activities. Recent work has demonstrated the screening of 100,000 molecules against the KDAC8 deacetylase (Gurard-Levin et al., ACS Comb Sci 13: 347-350 (2011)).

Modulators/Activators. As described herein, various aspects of the disclosure provide methods for screening activity of a library of oligonucleotide-functionalized spherical nucleic acids (SNAs) comprising: (a) individually contacting each SNA of the library with a cell, wherein upon contact with the SNA, the cell modulates expression of an enzyme and the amount of enzyme expressed is in proportion to the activity of the SNA; (b) contacting the enzyme expressed in step (a) with a substrate under conditions to transform the substrate to a product, wherein the product has a mass different from the substrate; (c) immobilizing the product and the substrate on a self-assembled monolayer (SAM) on a surface; (d) subjecting the immobilized substrate and product to mass spectrometry to produce a mass spectrum having a product signal and a substrate signal; and (e) correlating the product signal intensity to the substrate signal intensity to determine the extent of product formation and thereby assay the activity of each SNA. In some embodiments, the assay is performed in the presence of one or more potential modulators of the enzyme-substrate binding; subjecting the substrate and product to mass spectrometry to produce a mass spectrum having a product signal and a substrate signal; and binding of the enzyme and the substrate is detected by correlating a signal intensity of the product to a signal intensity of the substrate to determine the extent of product formation and thereby detecting the binding of the enzyme and the substrate in the presence of the one or more potential modulators.

In some embodiments, the modulator is an inhibitor of the enzyme and substrate binding. In further embodiments, the modulator is an activator of the enzyme and substrate binding.

### Variables in Creating the SNA Library

According to the methods of the disclosure, there are several variables that are available for modification when creating a SNA library. In various embodiments, the variables include but are not limited to structural properties (e.g., a SNA core property, an antigen property, an oligonucleotide property, or a combination thereof).

SNA core property. In some embodiments SNAs in a library vary by a property including but not limited to core diameter, core composition, or a combination thereof. In further embodiments, the core diameter is from about 30 nanometers (nm) to about 150 nm in mean diameter. In some embodiments, a plurality of SNAs is produced and the SNAs in the plurality have a mean diameter of less than or equal to about 50 nanometers (e.g., about 5 nanometers to about 50 nanometers, or about 5 nanometers to about 40 nanometers, or about 5 nanometers to about 30 nanometers, or about 5 nanometers to about 20 nanometers, or about 10 nanometers to about 50 nanometers, or about 10 nanometers to about 40 nanometers, or about 10 nanometers to about 30 nanometers, or about 10 nanometers to about 20 nanometers). In further embodiments, the SNAs in the plurality have a mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers.

In some embodiments, the core composition is 1,2-dioleoyl-sn-glycero-3-phosphocholine (DOPC), 1,2-di-(9Z-octadecenoyl)-sn-glycero-3-phosphoethanolamine (DOPE), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-dihexadecanoyl-sn-glycero-3-phosphoethanolamine (DPPE), or a combination thereof.

Antigens. In some embodiments, the disclosure contemplates that at least one SNA in the library further comprises an antigen. Variations in the antigen contemplated by the disclosure include antigen composition, antigen location, antigen density, or a combination thereof. In some embodiments, the antigen composition comprises human papillomavirus (HPV) E7 protein or ovalbumin (OVA). In further embodiments, the antigen location is encapsulated within the core or associated with the outer surface of the core. When the antigen is associated with the outer surface of the core, it is also contemplated that in some embodiments the antigen is associated with the oligonucleotide that is functionalized on the outer surface of the core, either through hybridization to an oligonucleotide attached to the core, or through direct attachment to the core.

The disclosure contemplates that, when making a library of SNAs, variation may be introduced in the relative amount of antigen that is encapsulated in one SNA in the library versus another SNA in the library. Thus, in various embodiments, the disclosure contemplates that at least two SNAs differ from each other in that one SNA comprises 2×, 3×, 4×, 5×, 6×, 7×, 8×, 9×, or 10× of encapsulated antigen relative to a second SNA. As used herein, the terms “2×” and “3×” etc. simply mean that one SNA comprises twice as much, or three times as much, etc., antigen relative to a second SNA.

The disclosure further contemplates that, when making a library of SNAs, variation may be introduced in the relative amount of antigen that is present on the outer surface of the SNA. As described herein, antigen may be associated with an oligonucleotide that is attached or associated with a SNA. Accordingly, in further embodiments, the disclosure contemplates that at least two SNAs differ from each other in that one SNA has 0%, 10%, 20%, 30%, 40%, 50%, 60%, 70%, 80%, 90%, or 100% of the oligonucleotide on the outer surface of the core associated with antigen relative to a second SNA.

Oligonucleotide properties. In some embodiments, the disclosure contemplates that SNAs in a library vary by a property including but not limited to oligonucleotide sequence, oligonucleotide conjugation chemistry, oligonucleotide conjugation terminus, oligonucleotide backbone, oligonucleotide density, complement density, or a combination thereof.

In some embodiments, the oligonucleotide sequence activates a Toll-like receptor (TLR). In further embodiments, the toll-like receptor is chosen from the group consisting of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13. In some embodiments, the TLR is TLR-9. In further embodiments, the oligonucleotide sequence comprises a CpG motif. Synthetic immunostimulatory oligonucleotides that contain CpG motifs that are similar to those found in bacterial DNA stimulate a similar response of the TLR receptors. Therefore immunomodulatory oligonucleotides have various potential therapeutic uses, including treatment of immune deficiency and cancer. Employment of liposomal nanoparticles functionalized with immunomodulatory oligonucleotides will allow for increased preferential uptake and therefore increased therapeutic efficacy. Thus, SNAs of the disclosure, functionalized with stabilized with functional CpG motif-containing nucleic acid, would provide enhanced therapeutic effect.

In some embodiments, the oligonucleotide conjugation chemistry is a cholesterol moiety. In further embodiments, the oligonucleotide conjugation chemistry is a cholesterol-modified oligonucleotide or a 1,2-di-(9Z-octadecenoyl)-sn-glycero-3-phosphoethanolamine (DOPE)-modified oligonucleotide. In still further embodiments, the oligonucleotide conjugation chemistry is a tocopherol moiety. In additional embodiments, the tocopherol is chosen from the group consisting of alpha-tocopherol, beta-tocopherol, gamma-tocopherol and delta-tocopherol.

Conjugation of an oligonucleotide with, e.g., a cholesterol or tocopherol moiety, is a further variable when creating a library of SNAs. In some embodiments, the oligonucleotide conjugation terminus is a 5′ terminus of the oligonucleotide or a 3′ terminus of the oligonucleotide.

In some embodiments, the oligonucleotide backbone affects the immunostimulatory activity of the SNA. Thus, in some embodiments the disclosure contemplates that the oligonucleotide backbone is a phosphodiester (PO) backbone or phosphorothioate (PS) backbone.

Density of oligonucleotide on the surface of the SNA is another variable that may be utilized when creating a library of SNAs. There are a variety of ways that oligonucleotide may be described. In some embodiments, the oligonucleotide surface density is about 0.5, about 1, or about 2 pmol/cm² (alternatively referred to herein as 1×, 2× and 4×, respectively)^{15,16}. In some embodiments, the oligonucleotide surface density is at least about 1 pmol/cm², or at least about 2 pmol/cm². In further embodiments, the oligonucleotide surface density is approximately 5 pmol/cm², 10 pmol/cm²,11 pmol/cm², 12 pmol/cm², 13 pmol/cm², 14 pmol/cm², 15 pmol/cm²,16 pmol/cm², 17 pmol/cm², 18 pmol/cm², 19 pmol/cm², 20 pmol/cm², or higher. In further embodiments, the oligonucleotide surface density is at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. Alternatively, the density of oligonucleotide on the surface of a SNA is measured by the number of oligonucleotides on the surface of the SNA. With respect to the oligonucleotide surface density on the surface of a SNA of the disclosure, it is contemplated that a SNA as described herein comprises from about 1 to about 100 oligonucleotides on its surface. In various embodiments, a SNA comprises from about 10 to about 100, or from 10 to about 90, or from about 10 to about 80, or from about 10 to about 70, or from about 10 to about 60, or from about 10 to about 50, or from about 10 to about 40, or from about 10 to about 30, or from about 10 to about 20 oligonucleotides on its surface. In further embodiments, a SNA comprises at least about 5, 10, 20, 30, 40, 50, 60, 70, 80, 90, or 100 oligonucleotides on its surface. In still further alternatives, and as discussed above, density may be referred to herein as 1×, 2×, etc. Thus, in some embodiments, at least two SNAs in the library differ from each other in that one SNA comprises a density of oligonucleotide on its outer surface that is 2×, 3×, 4×, 5×, 6×, 7×, 8×, 9×, or 10× that of a density of oligonucleotide on the outer surface of a second SNA. In still further alternatives, density is referred to in terms of how much of the oligonucleotide on the surface of the SNA is associated with a complementary oligonucleotide strand. Thus, in some embodiments, the disclosure contemplates that at least two SNAs differ from each other in that one SNA has 0%, 10%, 20%, 30%, 40%, 50%, 60%, 70%, 80%, 90%, or 100% of the oligonucleotide on the outer surface of the core associated with a complementary oligonucleotide relative to a second SNA.

### EXAMPLES

The following examples show how the methodology described herein was used to make and screen approximately 1000 (800 of which were unique) SNA architectures. In addition, it is described how machine learning models can be trained to predict immune activation from SNA structural considerations. Significantly, these models provide a ranking of the order of importance of eleven structural parameters and SNA drug concentration.

### Example 1

Materials. DOPE and DOPC were purchased from Avanti Lipids (Alabaster, Ala.).

Phosphoramidites for DNA synthesis were purchased from Glen Research (Sterling, Va.). Peptides were custom ordered from Genscript (Piscataway, N.J.). 2,2′-dipyridyldisulfide was purchased from Sigma Aldrich.

DNA Synthesis. DNA was synthesized with a MerMaid 12 synthesizer. Cholesterol modification was done on the column in the synthesizer. For DOPE-modified oligonucleotides, a thiol modified oligonucleotide was synthesized. DNA sequences are shown in Table 1, below.

TABLE 1
Oligonucleotide sequences used in this study.
Sp18 refers to the spacer 18 modifier
(Glen Research, Sterling, VA) and “X” is
either cholesteryl-TEG or thiol modifier.
Thiol-modified is converted to DOPE as
described herein. SH refers to thiol modifier.

| | | | SEQ |
|---|---|---|---|
| Name | Sequence (5′→3′) | Backbone | ID NO |
| ODN1826-3′ | TCC ATG ACG TTC CTG | PO and PS | 1 |
| Mod | ACG TT-SP18-SP18-X | | |
| ODN1826-5′ | X-SP18-SP18-TCC ATG | PO and PS | 2 |
| Mod | ACG TTC CTG ACG TT | | |
| GpC-ODN1826- | TCC ATG AGC TTC CTG | PO and PS | 3 |
| 3′ Mod | AGC TT-SP18-SP18-X | | |
| GpC-ODN1826- | X-SP18-SP18-TCC ATG | PO and PS | 4 |
| 5′ Mod | AGC TTC CTG AGC TT | | |
| Complement | AAC GTC AGG AAC GTC | PO | 5 |
| ODN1826- | ATG GA-SP18-SH | | |
| 3′ Mod | | | |
| Complement | SH-SP18-AAC GTC AGG | PO | 6 |
| ODN1826- | AAC GTC ATG GA | | |
| 5′ Mod | | | |

Synthesis of DOPE-SMPB. One mol equivalent of succinimidyl 4-(p-maleimidophenyl)butyrate (SMPB, Thermo-Fisher Scientific, Waltham, Mass.) and 1 mol equivalent of N,N-Diisopropylethylamine was added 1 mL of DOPE as received from Avanti Lipids (25 mg/mL in chloroform). The reaction was incubated for 24 hours at room temperature. The reaction was checked for completion with TLC using 20% methanol in dichloromethane as the mobile phase. Upon disappearance of the DOPE band in TLC, the reaction was washed three times with water and the organic phase was dried under a N2 stream.

DOPE Modification of Oligonucleotides. The thiol modified oligonucleotides was reduced with 200 mM DTT in 100 mM phosphate buffer (pH 8.0) for 2 hours at 40° C. The oligonucleotide was purified away from DTT with NAP-10 columns using water as the mobile phase (GE Healthcare, Chicago, Ill.). The reduced oligonucleotide was immediately reacted with DOPE-SMPB as follows. 50 equivalent of DOPE-SMPB was dissolved in ethanol in the same volume as the oligonucleotide. The two solutions were mixed together and incubated at room temperature for 24-48 hours. The reaction mixture was washed with chloroform three times to remove excess lipid. The interface and the aqueous phase was lyophilized. The reaction yield was determined by denaturing PAGE gels. Typically, yields were greater than 90% and no further cleanup was performed.

Synthesis of liposomes. 25 mg of DOPC in chloroform transferred to a glass vial and dried overnight into a thin film, first under a N2 stream followed by high vacuum. For DOPC-DOPE mixture liposomes, 20% by mol DOPE was added to the 25 mg of DOPC before drying. The lipid film was rehydrated with 1 mL of 1×PBS and vortexed until no more clumps were visible. For encapsulated peptides, the peptide was dissolved into the PBS at 0.1 and 1 mg/mL. The lipid suspensions were frozen in liquid nitrogen and thawed in a bath sonicator with sonication. The freeze thaw was repeated three times. The solution was then extruded through 200, 100, 80 and 50 nm filters. Two filters were used for each extrusion and the solution was passed through these filters 11 times. The liposomes were split into two after the 80 nm extrusion. Half of the solution was saved as the 80 nm liposomes, and the remainder was extruded through the 50 nm filter. The liposomes were dialyzed against 1×PBS overnight to remove non-encapsulated peptide. The liposomes were characterized by DLS for size (Z-average reported) and phosphatidylcholine assay for concentration (Millipore-Sigma, St. Louis, Mo.). DOPE did not interfere with the phosphatidylcholine assay, so it was assumed that the DOPE:DOPC ratio remained the same. The liposome concentrations were calculated from the diameter and the lipid concentration as described in Banga et a1³³.

Synthesis of complementary oligonucleotides with peptide. The complementary oligonucleotides were reduced with DTT as described above and mixed with 55 equivalents of 2,2′-dipyridyldisulfide in 100 mM phosphate buffer (pH 8.0). The reaction was incubated at 40° C. for 24 hours. The reaction process was monitored by absorption of pyridinethione at 343 nm. Upon completion, the modified oligonucleotide was washed three times with water in a 3K MWCO spin filter. The oligonucleotide was then mixed with 1 equivalent of C-OVA and incubated at 40° C. overnight again. The process was again monitored at 343 nm, and washed with a spin filter as described above.

Duplex formation. The purified peptide-oligonucleotide conjugate and 1 equivalent of the lipid-conjugated oligonucleotide was mixed in duplex buffer (30 mM HEPES (pH 7.4), 100 mM potassium acetate and 2 mM magnesium acetate). The mixture was heated to 65° C. for 10 minutes and slow cooled to room temperature.

SNA Synthesis. Lipid modified oligonucleotides or duplexed were mixed with liposomes in a 384 well plate in 40 μL final volume. The final concentration of lipid-modified oligonucleotide or duplex in each well was 10 μM. The concentration of liposomes was adjusted to accommodate SNAs of various oligonucleotide densities. After mixing, the plate was sealed and incubated at room temperature for 24 hours.

Synthesis of peptide substrate. The CRpY-NH2 peptide substrate was synthesized using standard fluorenylmethoxycarbonyl (Fmoc) solid phase peptide synthesis methods on a Rink-Amide resin. The N-terminus was acetylated. The peptide was purified by reverse phase HPLC on a C-18 column, in a gradient from water to acetonitrile and fractions were checked for the correct mass by MALDI-MS. The peptide was lyophilized and stored as a solid until use.

SAMDI plate and monolayer preparation. Stainless steel plates custom-designed for use in MALDI instruments were cleaned and used to evaporate a 1536-spot pattern of 5 nm Ti (0.02 nm/s), then 35 nm Au (0.05 nm/s), using an aluminum mask. The gold array plates were incubated overnight at 4° C. in an ethanolic solution containing a 1:4 ratio of an asymmetric disulfide terminated with a maleimide group and a tri(ethylene glycol) group and a symmetric disulfide terminated with tri(ethylene glycol) groups, with a 0.5 mM total disulfide concentration. The plates were then rinsed with ethanol, dried, and placed in a solution of 10 mM hexadecyl phosphonic acid in ethanol for 10 min at room temperature. Plates were then rinsed with ethanol and dried and used for the SEAP assay.

SEAP assay. RAW-Blue cells (Invivogen) were cultured as described by the manufacturer. The cells were collected and suspended at 550,000 cells/mL, and 17,000 cells were distributed into 384-well plate culture plates with a Thermo Scientific Multidrop Combi. 10×SNA solutions were added to the cell culture plates with a Tecan liquid handler, then cultured at 37° C. and 5% CO₂. After approximately 16 hours, the cell culture plates were centrifuged at 300 crf for 1 minute, then 10 μL of media was transferred to a 384-well reaction plate. Recombinant SEAP (0-1,600 ng/mL) was prepared in media from untreated cells and was added to empty wells, and was used as the standard curve. To minimize free thiols in the media, which compete with the substrate immobilization, 1 μL of 11 mM TCEP in water was added and incubated for 15 minutes at 60° C. to the plates to first reduce cystine to cysteine. The 60° C. incubation also inactivates any potential phosphatases other than SEAP, which is stable at 60° C. Next, 1 μL of 12 mM maleimide was added to react with free cysteines for 1 hour, 37° C. 8 μL of 75 μM CRpY peptide substrate in reaction buffer (300 mM Tris, pH 8.5, 2.5 mM MgCl₂) was added to the reaction plate, then incubated for 1 hour at 37° C. 2 μL of 11 mM pridoxal 5′-phosphate hydrate in reaction buffer was added. The 0.75 μL of the reaction solutions were transferred to 1536-spot SAMDI array plates and incubated for 1 hour at 37° C. The plates were rinsed with water and ethanol, then dried with air. Matrix (15 mg/mL 2,4,6-trihydroxyacetophenone in acetone) was applied to the SAMDI plates, where were analyzed by MALDI using an AB Sciex 5800 MALDI TOF/TOF instrument in positive reflector mode. The spectra were analyzed by calculating the area under the curves for the [M+H]⁺ and [M+Na]⁺ disulfide peaks corresponding to the substrate and product masses, using custom software. Each SNA subset was tested in two wells (biological replicates) and each sample was tested on two SAMDI spots (technical replicates). Technical replicates with sub-threshold signal-to-noise were excluded from analysis.

Quantitative structure-activity relationship (QSAR) model. QSAR models were trained to predict immune activation from SNA properties. The training data contained 336 SNA rows with 9 property columns for datasets 1 and 2 and 288 SNA rows with 8 property columns for dataset 3. The response vector, also called predicted variable, is the immune activation measured via SEAP concentration. Cross-validation was used, where a sample of data is left out for model testing, to calculate the predictive power Q² metric:

$${Q}^{2} = 1 - \frac{{\sum }_{i}^{n} {( {y}_{i} - {\hat{y}}_{i} )}^{2}}{{\sum }_{i}^{n} {( {y}_{i} - {\overline{y}}_{t r a i n} )}^{2}}$$

In this formulation, yᵢ is the immune activation for test SNA i, ŷ_{ι} is the predicted immune activation, y̅ₜᵣₐᵢₙ is the immune activation of the training set, and n is the number of cross-validated test SNAs. The Q² metric can take on values from −∞ to 1, where 1 is perfect prediction and 0 is equivalent to random performance, which predicts the mean immune activation of all SNAs in the training set. Five-fold cross validation was used, where a random 80% of the data is selected for training, with the remaining 20% as validation. Three models were selected to test for linear relationships amongst SNA properties and immune activation: linear regression, logistic regression, and the non-linear model xgboost. If all relationships were linear, then Q² would be similar for all models. Similarly, logistic regression can fit trends that are more complex than linear regression, but it still treats multiple properties as linearly related and is still a linear model. For all models, an explicit null model was created by randomizing the data values prior to model training.

Statistics. Multi-way ANOVA was performed on each SNA subset, using MATLAB software. Statistical comparisons of paired data were made using the two-tailed Wilcoxon test; unpaired data was compared with a two-tailed t-test. All error bars in figures represent standard error of the mean.

### Example 2

Three subsets of SNAs (OVA encapsulated SNAs, E7 encapsulated SNAs and surface-presented OVA SNAs) are tested herein, representing the key possible combinations of the parameters, with a few synthesis-limited exceptions noted below regarding lipid composition, oligonucleotide surface density, and surface conjugated peptide antigen (see below and FIG. 1c).

Here, media containing SEAP was mixed with a phosphorylated peptide substrate, captured the substrate and dephosphorylated product on monolayers and analyzed the samples by SAMDI. This platform was chosen for its ability to quantify enzyme activities at high-throughput, without dependence on optical methods of measurement. Optical measurement techniques can be negatively affected by the light scattering and absorbance of the nanoparticles, which are difficult to correct for because of their dependence on nanoparticle properties such as size and concentration. Furthermore, SAMDI requires small sample volumes for analysis, thereby reducing the amount of SNAs, cells, and reagents necessary for evaluation.

### SNAs Induce Higher Immune Activation than Linear Oligonucleotides

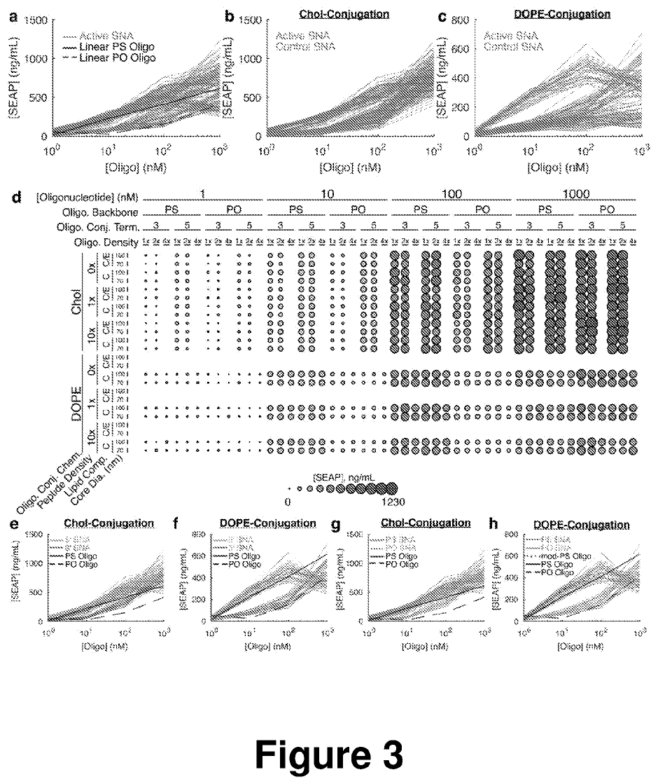

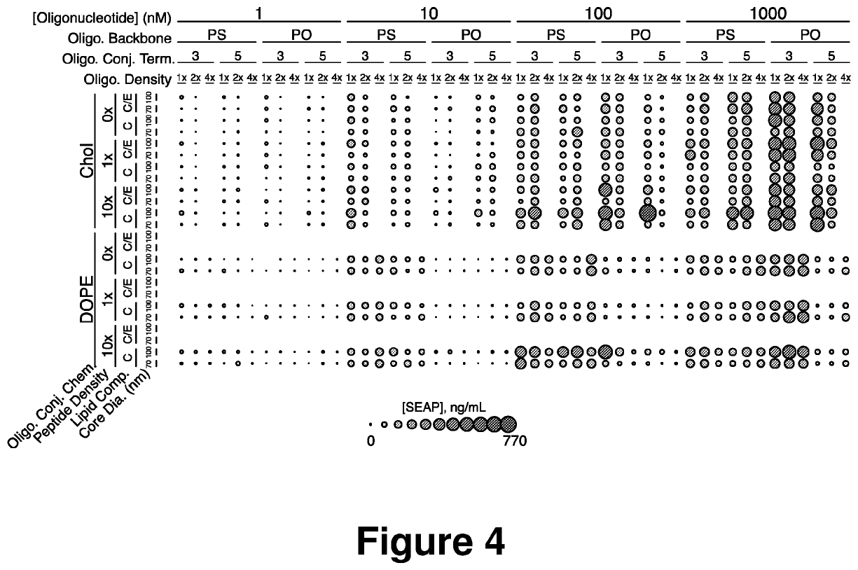

First, the immune activation of SNAs was compared to linear PO and PS oligonucleotides. The SNAs induced a broad range of immune activation (FIGS. 3a and 3d show the encapsulated OVA subset with the active CpG oligonucleotide sequence; FIG. 4 shows the encapsulated E7 subset). Almost all SNAs with the active oligonucleotide sequence outperformed the linear PO oligonucleotide, and many SNAs, including ones with the PO backbone, outperformed the linear oligonucleotide with the PS backbone.

### Conjugation Chemistry Significantly Affects Immune Activation by SNAs

To understand which properties had a significant impact on immune activation, a multifactor analysis of variance (ANOVA) (Table 2) was performed on the encapsulated antigen SNA data subsets, which revealed that after oligonucleotide concentration and oligonucleotide sequence (i.e., active or control), oligonucleotide conjugation chemistry had the greatest impact on immune activation. Cholesterol conjugation resulted in higher levels of immune activation than DOPE conjugation (P=4.8×10⁻¹⁶). However, SNAs with cholesterol-conjugated control oligonucleotides also induced similarly high levels of activation at 1000 nM oligonucleotide concentration (798 and 747 ng/mL SEAP for active and inactive, respectively—FIG. 3b). Since the control linear oligonucleotide does not activate TLR9 signaling, these results indicated that these SNAs activate NF-κB through another mechanism. One possible explanation is that cholesterol induces additional activation. Our cholesterol conjugation chemistry utilizes carbamates, which can be cleaved by esterases, including sterol O-acyltransferases²³. Any potentially released cholesterol, which is known to activate the UPR pathway in macrophages, may also induce NF-κB activation²⁴.

In contrast, SNAs with control oligonucleotides conjugated to DOPE lead to dramatically lower SEAP secretion compared to their cholesterol-conjugated counterparts (P<1×10⁻¹⁶, FIG. 3c). From these results, it was concluded that DOPE conjugation provides an advantage if targeted TLR9 activation is exclusively desired. However, the combination of TLR9 stimulation and non-specific activation by SNAs with cholesterol-conjugated oligonucleotides may be advantageous for inducing a greater overall immune response.

TABLE 2
Multi-factor ANOVA of three SNA subsets.

| | ENCAPSULATED | | | ENCAPSULATED | | | SURFACE-PRESENTED | | |
|---|---|---|---|---|---|---|---|---|---|
| | OVA SUBSET | | | E7 SUBSET | | | OVA SUBSET | | |
| FACTOR | D.F. | F | P | D.F. | F | P | D.F | F | P |
| CONCENTRATION | 3 | 1240 | <1E−220 | 3 | 412 | 2.5E−220 | 3 | 183 | 4.7E−106 |
| SEQUENCE | 1 | 381 | 2.0E−79 | 1 | 261 | 3.6E−56 | 1 | 246 | 1.2E−52 |
| CONJ. CHEM. | 1 | 338 | 4.3E−71 | 1 | 103 | 6.0E−24 | N/A | N/A | N/A |
| BACKBONE | 1 | 22.6 | 2.1E−06 | 1 | 3.64 | 0.056 | 1 | 241 | 8.5E−52 |
| CONJ. TERM. | 1 | 32.6 | 1.3E−08 | 1 | 3.34 | 0.068 | 1 | 2.73 | 0.099 |
| OLIGO. DENS. | 2 | 5.59 | 0.0038 | 2 | 11.5 | 1.0E−05 | 2 | 2.23 | 0.11 |
| ANTIGEN DENS. | 2 | 0.945 | 0.39 | 2 | 33.2 | 5.6E−15 | 2 | 0.673 | 0.51 |
| LIPID COMP. | 1 | 2.17 | 0.14 | 1 | 0.0839 | 0.77 | N/A | N/A | N/A |
| CORE DIAMETER | 1 | 0.0248 | 0.87 | 1 | 20.4 | 6.6E−06 | 1 | 0.0218 | 0.88 |
| COMP. DENS. | N/A | N/A | N/A | N/A | N/A | N/A | 2 | 1.34 | 0.26 |

### Conjugation Terminus of the Oligonucleotide Influences the Immune Activation in a Conjugation Chemistry Dependent Manner

Due to the dominant effects of conjugation chemistry, the remaining SNA properties were analyzed separately for SNAs with cholesterol- and DOPE-conjugated oligonucleotides. Interestingly, differences in conjugation terminus were observed with different conjugation chemistries (FIGS. 3e and f). With cholesterol conjugation, 5′ conjugated SNAs showed significantly different activity than 3′ conjugated SNAs (OVA subset: P<2.2×10⁻¹⁶ for all concentrations; 566 and 439 ng/mL mean SEAP at 100 nM for 5′ and 3′ conjugation, respectively), but DOPE-conjugated SNAs did not show a difference with conjugation terminus (OVA subset: P=1 for all concentrations; 324 and 330 ng/mL mean SEAP at 100 nM for 5′ and 3′ conjugation, respectively). In addition, conjugation from the 5′ terminus did not lead to loss of immune activation for either conjugation chemistry, which contradicts reports that modifications at the 5′ end inactivate the TLR9 activity of linear CpG oligonucleotides^{25,26}.

### Phosphorothioate Oligonucleotide Backbone Increases Immune Activation Compared to Phosphodiester Backbone

Oligonucleotide backbone also influenced immunostimulatory activity of SNAs (Table 2 and FIGS. 3g, h, and i). Generally, SNAs with PS backbones outperformed their PO counterparts (P=5×10⁻⁹ for DOPE and P=2.7×10⁻⁴ for cholesterol-conjugated SNAs), which is consistent with reported trends observed for linear oligonucleotides¹⁴. However, a more pronounced dependence on oligonucleotide backbone was observed with DOPE-conjugated SNAs than with cholesterol-conjugated SNAs. For DOPE-conjugated SNAs, the mean SEAP concentrations were 191 and 463 ng/mL for PO and PS backbones, respectively, whereas for cholesterol-conjugated SNAs, it was 431 and 573 ng/mL at 100 nM).

In contrast, SNAs with PO oligonucleotides outperformed their PS counterparts at the highest concentration of 1000 nM. Notably, the activity induced by DOPE-conjugated SNAs with PS oligonucleotides consistently decreased when the oligonucleotide concentration was increased from 100 nM to 1000 nM. The DOPE-modified PS linear oligonucleotide, but not the unmodified version, showed a similar reduction in activity at 1000 nM (FIG. 3h), suggesting that this behavior is due to the specific stimulatory properties of the modified oligonucleotide.

These results lead to the conclusion that DOPE-conjugated oligonucleotides with PS backbones provide an advantage if greater potency is desired. PS backbones have the added benefit of resistance to nuclease degradation in vivo²⁷. However, these results also show that SNAs with oligonucleotides composed of PO backbones can achieve similar levels of activation, though at higher concentrations. While class B CpG oligonucleotides are less effective with PO backbones, using SNAs with PO oligonucleotides may be worth the loss in potency because of the reduction in both toxicity and side effects, since the SNA structure may provide sufficient resistance to nuclease activity.

### Oligonucleotide Density on the Surface of the Nanoparticle has a Small and Variable Impact on Immune Activation

Surprisingly, there was not a strong or consistent trend showing either the lowest or highest oligonucleotide densities as the most effective designs. In previous studies, SNAs with higher oligonucleotide densities led to higher biological activity, such as uptake and RNAse H mediated degradation of mRNA; however, the nanoparticle designs in these studies were limited to a small parameter space with multiple differences (e.g., gold cores, different core sizes and oligonucleotide densities)^{15,16}. From these observations, it was concluded that the selection of oligonucleotide density based on other considerations, such as stability in vivo, is essential since all densities tested here show approximately equal efficacy.

### Core Diameter and Lipid Composition Influences the Immune Activation of SNAs in an Encapsulated Peptide-Specific Manner

In both encapsulated subsets, lipid composition generally did not have a significant impact (Table 2), except for in a particular context discussed below. Core diameter was not significant in the encapsulated OVA subset, whereas it had a significant impact with encapsulated E7.

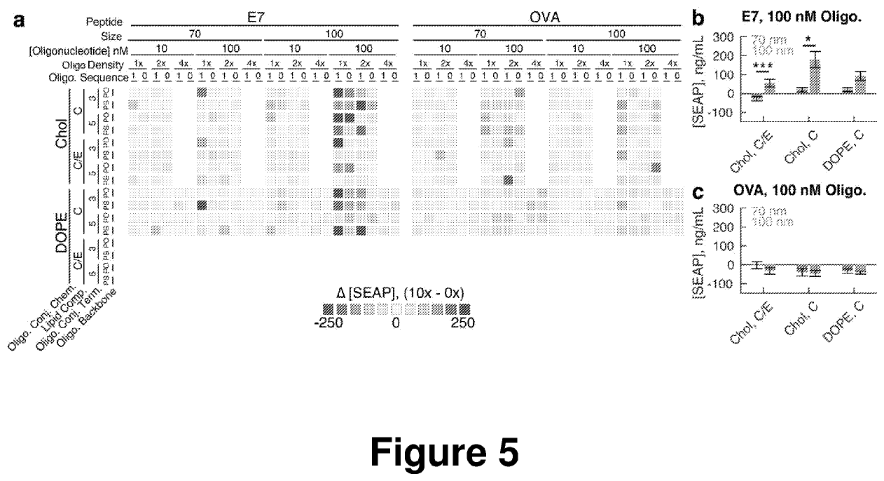

To isolate the effects of encapsulating the peptides, the differences of each SNA variant with and without peptide was investigated. SNAs with identical properties except for the amount of peptide encapsulation were paired, and then the SEAP concentration of the SNA without peptide was subtracted from the SNA with 10× peptide (FIG. 5a). For the E7 subset, it was observed that SNAs with 100 nm cores containing 10× peptide induced higher levels of NF-κB activation (P=5.7×10⁻⁵), and the magnitude of this effect depended on lipid composition as well (FIG. 5b). Within cholesterol-conjugated SNAs with 100 nm cores, the SNAs with 100% DOPC cores showed higher immune activation than 80% DOPC, 20% DOPE cores (P=0.0011) (FIG. 5b). However, for the OVA subset, there was no difference between SNAs with different core sizes or lipid compositions (FIG. 5c).

These results illustrated that peptide encapsulation can impact the efficacy of SNAs, positively or negatively, and that the impact is dependent on other SNA design properties as well. Unlike oligonucleotides, the physicochemical properties of peptides vary dramatically with sequence, which can affect their interaction with the rest of the SNA structure. For example, one possibility is that the differences in isoelectric points of the peptides, which are 5.7 and 8.8 for the E7 and OVA peptides, respectively, result in different net charges for the peptides, which could affect their interaction with the positively charged liposome core. It was concluded that the interactions between liposomes and peptides have to be taken into account when synthesizing nanomedicines, as they can lead to large shifts in the immune activation of SNAs, especially at high concentrations of peptide encapsulation.

### Example 3

### Effects of Hybridization of Complementary Strands onto the SNAs

Next, an alternative method for incorporating the antigen into the SNA design was investigated. A cysteine-modified OVA peptide was conjugated to a thiol-modified oligonucleotide complementary to the SNA-conjugated oligonucleotide, the two oligonucleotides were hybridized to form a duplex and SNAs were synthesized that present this duplex on the surface. To separate the impact of the hybridized complement from the conjugated peptide, SNAs with the complement but without the peptide were also synthesized. In this conjugated OVA subset, DOPE-conjugated oligonucleotides were used to prevent the non-specific NF-κB activation by cholesterol-conjugated SNAs described above.

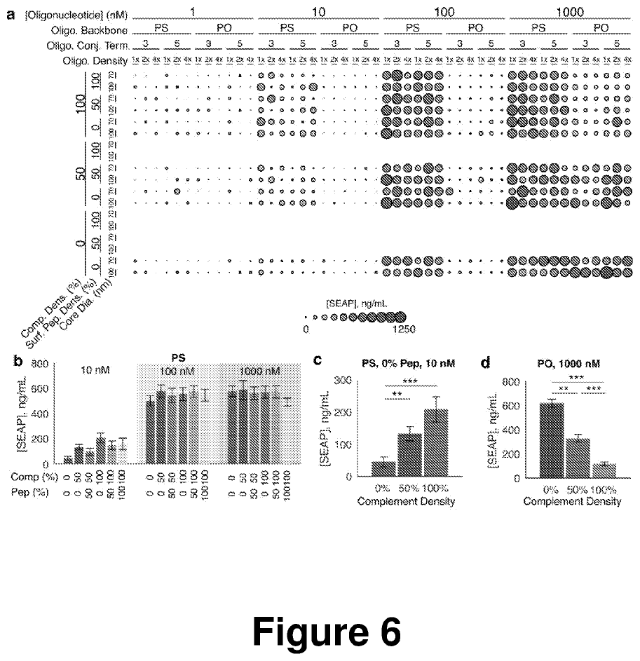

Results showed that SNAs synthesized with this strategy shared some trends with their single-stranded counterparts. It was observed that after oligonucleotide sequence, the most influential property on immune activation was backbone chemistry, with PS backbones outperforming PO versions (FIG. 6a). Again, the core properties of lipid composition and core diameter were not significant.

Interestingly, for the SNAs with PS oligonucleotides, the addition of the complement oligonucleotide, either to half or to all of the anchored oligonucleotides, did not change immune activation at concentrations of 100 or 1000 nM, respectively (FIG. 6b). Furthermore, there was no difference between SNAs composed of the complement, with and without conjugated peptide. Interestingly, it was observed that higher complement densities led to higher immune activation at 10 nM. This effect may be a function of SNA uptake, where higher complement densities create higher charge densities on the surface and increase the uptake of SNAs, which in turn lead to higher immune activation. However, the opposite trend was observed with PO oligonucleotides at 1000 nM, where duplexing reduced activity. A possible explanation for the decreased activity in duplexed SNAs is that the duplexing interferes with the oligonucleotide interaction with TLR9, however, it is not clear why this interaction with TLR9 would be different with PO and PS backbones. These results suggested that the strategy of including antigens by duplexing antigen-conjugated complementary oligonucleotides is effective with PS SNAs without concern for losing activation of TLR9.

### Example 4

### Supervised Machine Learning Captures Non-Linearity of Property Interactions and Confirms Trends in Biological Importance of Properties

Three supervised learning models (linear regression, logistic regression, and non-linear xgboost) were trained to predict immune activation and to evaluate the relationships between SNA properties and confirm their relative impact^{28,29}. The predictive power of the models was quantified with the Q² statistic, which describes how close the predicted SEAP concentrations are to the measured values. Q² ranges from −∞ to 1, where 0 indicates no predictive power, equivalent to predicting the mean, and 1 indicates perfect prediction³⁰.

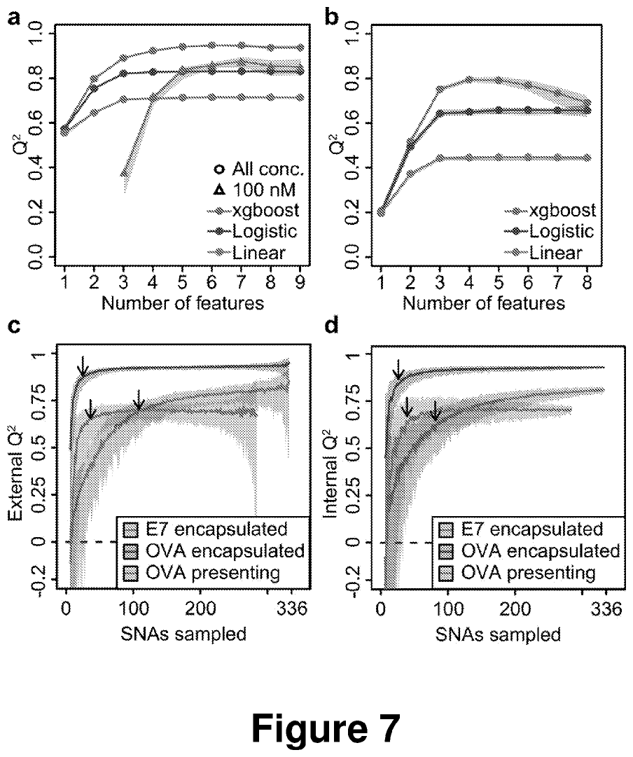

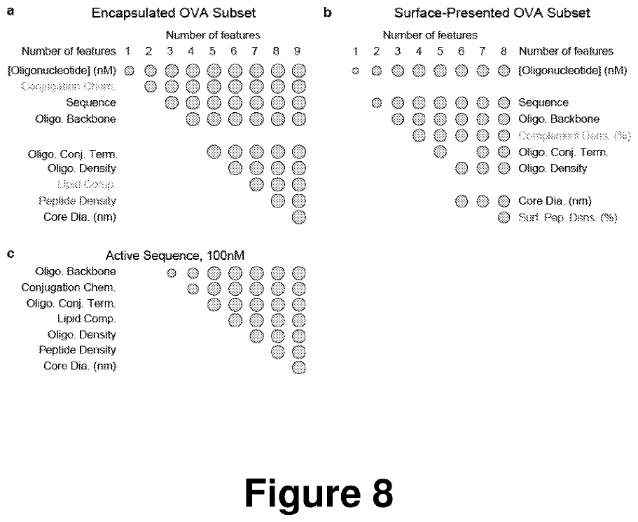

Each model was trained with all combinations of properties (e.g., all pairs, all triplets, etc.) and their Q² performance was analyzed. As additional properties were added to the models, the Q² performance increased, plateauing for most models and decreasing in the xgboost model for the surface-presented OVA subset (FIGS. 7a and b). Since clear non-linear trends were observed in the data as described above, the model performance increased with the non-linearity of the model in both subsets (mean increase from 0.53 to 0.83 for xgboost). Analysis of the most predictive SNA property combinations demonstrate that highly predictive properties remain significant and informative as more properties are introduced into the model (FIGS. 8a and b). In addition, the order of importance of properties was largely consistent between the encapsulated OVA and the surface-conjugated OVA subsets, suggesting that the ordering is robust regardless of peptide localization.

For the encapsulated OVA and surface-presented OVA subsets, the Q² value stops increasing beyond five and four properties, respectively (FIG. 7a). At first glance one might conclude that only these highly predictive properties are relevant; however, when repeating this analysis with fixed values for sequence and concentration (the two features with the greatest impact), Q² values stop increasing after another five properties (FIG. 7a), indicating that formerly seemingly non-predictive properties do, in fact, influence immune activation (FIG. 8c). Taken together, these properties, which appear non-influential in a global context, become impactful in a restricted design space.

### Capturing Maximal SAR with Minimal SNA Synthesis and Evaluation

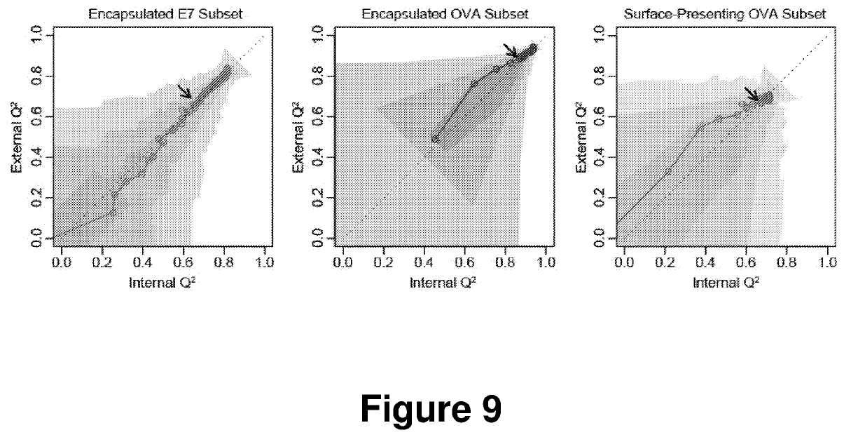

Next, it was investigated whether a similar Q² level is attainable with fewer randomly selected SNA designs. This question is particularly relevant when synthesis and evaluation of full libraries are impractical, but where exploration of a large design space is desired. In that case, one could synthesize a random subset, while capturing most of the trends. To this end, this process was simulated by training an xgboost model on a random selection of SNAs and testing predictions on the remaining, unselected SNAs within the three subsets (FIG. 7c). The point of diminishing returns was identified, which balances the minimum number of SNAs with maximum Q², by calculating the sample size closest to training size 1 and Q²=1. This point was 90, 20, and 31 SNAs (out of 336, 336, and 288 SNAs) with Q²=0.67, 0.88, and 0.66 for encapsulated E7, encapsulated OVA, and surface-presented OVA subsets, respectively. These points represent a mean of 16% of the total number of SNAs, suggesting that a small number of randomly selected SNAs can predict SAR of a relatively large SNA library. In practice, this external Q² (prediction of non-synthesized SNAs) cannot be measured with a randomized subsample, but an internal Q² can be measured by cross-validating within the randomized subsample. It is shown herein that the internal and external Q² are highly correlated (FIG. 7d and FIG. 9), suggesting that the point of diminishing returns can be identified as SNAs are continually synthesized from an arbitrary library size. Combined with high-throughput SNA synthesis described above, the machine learning analysis showed that a combined experimental/computational method can probe and predict the SAR of tens of thousands of SNAs with a much smaller subset of structures (e.g., approximately 1000).

### CONCLUSIONS

The above examples, when combined with other high throughput approaches pioneered by Anderson and Langer, makes the case for the need to consider the vast possibility of structure-property relationships in designing nanomedicines^{31,32}. Moreover, it shows that properties can be strongly interrelated, and it emphasizes the danger in making global conclusions about one structural consideration being more critical than others. This interdependence and non-linearity are underscored when applying the non-linear machine-learning models, as opposed to linear ones, in predicting the biological response of SNAs. Indeed, to realize rational approaches to vaccinology, this work makes a strong case for the combination of high-throughput experimentation and computational analysis, in determining the structure-activity relationships of nanomedicines in general and SNAs in particular.

### REFERENCES

1 Bobo, D., Robinson, K. J., Islam, J., Thurecht, K. J. & Corrie, S. R. Nanoparticle-Based Medicines: A Review of FDA-Approved Materials and Clinical Trials to Date. Pharmaceutical research 33, 2373-2387, doi:10.1007/s11095-016-1958-5 (2016).
2 Mirkin, C. A., Letsinger, R. L., Mucic, R. C. & Storhoff, J. J. A DNA-based method for rationally assembling nanoparticles into macroscopic materials. Nature 382, 607-609, doi:10.1038/382607a0 (1996).
3 Cutler, J. I., Auyeung, E. & Mirkin, C. A. Spherical nucleic acids. Journal of the American Chemical Society 134, 1376-1391, doi:10.1021/ja209351u (2012).
4 Choi, C. H. J., Hao, L., Narayan, S. P., Auyeung, E. & Mirkin, C. a. Mechanism for the endocytosis of spherical nucleic acid nanoparticle conjugates. Proceedings of the National Academy of Sciences of the United States of America 110, 7625-7630, doi:10.1073/pnas.1305804110 (2013).
5 Radovic-Moreno, A. F. et al. Immunomodulatory spherical nucleic acids. Proceedings of the National Academy of Sciences 112, 3892-3897, doi:10.1073/pnas.1502850112 (2015).
6 Rosi, N. L. et al. Oligonucleotide-modified gold nanoparticles for intracellular gene regulation. Science (New York, N.Y.) 312, 1027-1030, doi:10.1126/science.1125559 (2006).
7 Seferos, D. S., Prigodich, A. E., Giljohann, D. A., Patel, P. C. & Mirkin, C. A. Polyvalent DNA nanoparticle conjugates stabilize nucleic acids. Nano Letters 9, 308-311, doi:10.1021/nl802958f (2009).
8 Li, J. et al. A review on phospholipids and their main applications in drug delivery systems. Asian Journal of Pharmaceutical Sciences 10, 81-98, doi:https://doi.org/10.1016/j.ajps.2014.09.004 (2015).
9 Schroit, A. J., Madsen, J. & Nayar, R. Liposome-cell interactions: in vitro discrimination of uptake mechanism and in vivo targeting strategies to mononuclear phagocytes. Chemistry and physics of lipids 40, 373-393 (1986).
10 Simoes, S., Slepushkin, V., Duzgunes, N. & Pedroso de Lima, M. C. On the mechanisms of internalization and intracellular delivery mediated by pH-sensitive liposomes. Biochim Biophys Acta 1515, 23-37 (2001).
11 McCluskie, M. J. & Davis, H. L. CpG DNA as mucosal adjuvant. Vaccine 18, 231-237 (1999).
12 Krieg, A. M. et al. CpG motifs in bacterial DNA trigger direct B-cell activation. Nature 374, 546-549, doi:10.1038/374546a0 (1995).
13 Hemmi, H. et al. A Toll-like receptor recognizes bacterial DNA. Nature 408, 740-745, doi:10.1038/35047123 (2000).
14 Zhao, Q., Temsamani, J., ladarola, P. L., Jiang, Z. & Agrawal, S. Effect of different chemically modified oligodeoxynucleotides on immune stimulation. Biochemical pharmacology 51, 173-182 (1996).
15 Giljohann, D. A. et al. Oligonucleotide loading determines cellular uptake of DNA-modified gold nanoparticles. Nano Letters 7, 3818-3821, doi:10.1021/nl072471q (2007).
16 Prigodich, A. E., Alhasan, A. H. & Mirkin, C. A. Selective enhancement of nucleases by polyvalent DNA-functionalized gold nanoparticles. Journal of the American Chemical Society 133, 2120-2123 (2011).
17 Gendron, K. B., Rodriguez, A. & Sewell, D. A. Vaccination with human papillomavirus type 16 E7 peptide with CpG oligonucleotides for prevention of tumor growth in mice. Archives of otolaryngology—head & neck surgery 132, 327-332, doi:10.1001/archotol.132.3.327 (2006).
18 Berns, E. J., Cabezas, M. D. & Mrksich, M. Cellular Assays with a Molecular Endpoint Measured by SAMDI Mass Spectrometry. Small 12, 3811-3818, doi:10.1002/smll.201502940 (2016).
19 Min, D. H., Tang, W. J. & Mrksich, M. Chemical screening by mass spectrometry to identify inhibitors of anthrax lethal factor. Nature biotechnology 22, 717-723, doi:10.1038/nbt973 (2004).
20 Mrksich, M. Mass spectrometry of self-assembled monolayers: a new tool for molecular surface science. ACS Nano 2, 7-18 (2008).
21 Su, J. & Mrksich, M. Using mass spectrometry to characterize self-assembled monolayers presenting peptides, proteins, and carbohydrates. Angewandte Chemie 41, 4715-4718, doi:10.1002/anie.200290026 (2002).
22 Su, J., Rajapaksha, T. W., Peter, M. E. & Mrksich, M. Assays of endogenous caspase activities: a comparison of mass spectrometry and fluorescence formats. Anal Chem 78, 4945-4951, doi:10.1021/ac051974i (2006).
23 Humerickhouse, R., Lohrbach, K., Li, L., Bosron, W. F. & Dolan, M. E. Characterization of CPT-11 hydrolysis by human liver carboxylesterase isoforms hCE-1 and hCE-2. Cancer Res 60, 1189-1192 (2000).
24 Li, Y. et al. Free cholesterol-loaded macrophages are an abundant source of tumor necrosis factor-alpha and interleukin-6: model of NF-kappaB- and map kinase-dependent inflammation in advanced atherosclerosis. J Biol Chem 280, 21763-21772, doi:10.1074/jbc.M501759200 (2005).
25 Yu, D., Zhao, Q., Kandimalla, E. R. & Agrawal, S. Accessible 5′-end of CpG-containing phosphorothioate oligodeoxynucleotides is essential for immunostimulatory activity. Bioorganic & medicinal chemistry letters 10, 2585-2588 (2000).
26 Kandimalla, E. R. et al. Conjugation of ligands at the 5′-end of CpG DNA affects immunostimulatory activity. Bioconjug Chem 13, 966-974 (2002).
27 De Clercq, E., Eckstein, E. & Merigan, T. C. [Interferon induction increased through chemical modification of a synthetic polyribonucleotide]. Science 165, 1137-1139 (1969).
28 Chen, T. & Guestrin, C. in Proceedings of the 22nd ACM SIGKDD International Conference on Knowledge Discovery and Data Mining 785-794 (ACM, San Francisco, Calif., USA, 2016).
29 Menard, S. Applied Logistic Regression Analysis. Vol. 106 (Sage, 2002).
30 Schuurmann, G., Ebert, R. U., Chen, J., Wang, B. & Kuhne, R. External validation and prediction employing the predictive squared correlation coefficient test set activity mean vs training set activity mean. Journal of chemical information and modeling 48, 2140-2145, doi:10.1021/ci800253u (2008).
31 Akinc, A. et al. A combinatorial library of lipid-like materials for delivery of RNAi therapeutics. Nature biotechnology 26, 561-569, doi:10.1038/nbt1402 (2008).
32 Anderson, D. G., Lynn, D. M. & Langer, R. Semi-automated synthesis and screening of a large library of degradable cationic polymers for gene delivery. Angewandte Chemie (International ed. in English) 42, 3153-3158, doi:10.1002/anie.200351244 (2003).
33 Banga, R. J., Chernyak, N., Narayan, S. P., Nguyen, S. T. & Mirkin, C. A. Liposomal Spherical Nucleic Acids. Journal of the American Chemical Society 136, 9866-9869, doi:10.1021/ja504845f (2014).

What is claimed is:

1. A method of screening activity of a library of oligonucleotide-functionalized spherical nucleic acids (SNAs) comprising:
(a) individually contacting each SNA of the library with a cell, wherein upon contact with the SNA, the cell modulates expression of an enzyme and the amount of enzyme expressed is in proportion to the activity of the SNA;
(b) contacting the enzyme expressed in step (a) with a substrate under conditions to transform the substrate to a product, wherein the product has a mass different from the substrate;
(c) immobilizing the product and the substrate on a self-assembled monolayer (SAM) on a surface;
(d) subjecting the immobilized substrate and product to mass spectrometry to produce a mass spectrum having a product signal and a substrate signal; and
(e) correlating the product signal intensity to the substrate signal intensity to determine the extent of product formation and thereby assay the activity of each SNA.

2. The method of claim 1, wherein at least one SNA in the library further comprises an antigen.

3. The method of claim 2, wherein the SNAs of the library differ in at least one structural parameter, and the structural parameter is a SNA core property, an antigen property, an oligonucleotide property, or a combination thereof.

4. The method of claim 3, wherein the SNA core property is core diameter, core composition, or a combination thereof.

5. The method of claim 4, wherein the core diameter is from about 30 nanometers (nm) to about 150 nm in mean diameter.

6. The method of claim 4, wherein the core composition is 1,2-dioleoyl-sn-glycero-3-phosphocholine (DOPC), 1,2-di-(9Z-octadecenoyl)-sn-glycero-3-phosphoethanolamine (DOPE), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-dihexadecanoyl-sn-glycero-3-phosphoethanolamine (DPPE), or a combination thereof.

7. The method of claim 3, wherein the antigen property is antigen composition, antigen location, antigen density, or a combination thereof.

8. The method of claim 7, wherein the antigen composition comprises human papillomavirus (HPV) E7 protein or ovalbumin (OVA).

9. The method of claim 7 or claim 8, wherein the antigen location is encapsulated within the core or associated with the outer surface of the core.

10. The method of claim 9, wherein the antigen is associated with the oligonucleotide that is functionalized on the outer surface of the core.

11. The method of claim 9, wherein the at least two SNAs differ from each other in that one SNA comprises 2×, 3×, 4×, 5×, 6×, 7×, 8×, 9×, or 10× of encapsulated antigen relative to a second SNA.

12. The method of claim 10, wherein the at least two SNAs differ from each other in that one SNA has 0%, 10%, 20%, 30%, 40%, 50%, 60%, 70%, 80%, 90%, or 100% of the oligonucleotide on the outer surface of the core associated with antigen relative to a second SNA.

13. The method of any one of claims 3-12, wherein the oligonucleotide property is oligonucleotide sequence, oligonucleotide conjugation chemistry, oligonucleotide conjugation terminus, oligonucleotide backbone, oligonucleotide density, complement density, or a combination thereof.

14. The method of claim 13, wherein the oligonucleotide sequence activates a Toll-like receptor (TLR).

15. The method of claim 14, wherein the TLR is TLR-9.

16. The method of claim 14 or claim 15, wherein the oligonucleotide sequence comprises a CpG motif.

17. The method of any one of claims 13-16, wherein the oligonucleotide conjugation chemistry is a cholesterol-modified oligonucleotide or a 1,2-di-(9Z-octadecenoyl)-sn-glycero-3-phosphoethanolamine (DOPE)-modified oligonucleotide.

18. The method of any one of claims 13-17, wherein the oligonucleotide conjugation terminus is a 5′ terminus of the oligonucleotide or a 3′ terminus of the oligonucleotide.

19. The method of any one of claims 13-18, wherein the oligonucleotide backbone is a phosphodiester (PO) backbone or phosphorothioate (PS) backbone.

20. The method of any one of claims 13-19, wherein the at least two SNAs differ from each other in that one SNA comprises a density of oligonucleotide on its outer surface that is 2×, 3×, 4×, 5×, 6×, 7×, 8×, 9×, or 10× that of a density of oligonucleotide on the outer surface of a second SNA.

21. The method of any one of claims 13-20, wherein the at least two SNAs differ from each other in that one SNA has 0%, 10%, 20%, 30%, 40%, 50%, 60%, 70%, 80%, 90%, or 100% of the oligonucleotide on the outer surface of the core associated with a complementary oligonucleotide relative to a second SNA.

22. The method of any one of claims 1-21, wherein each SNA in the library of oligonucleotide-functionalized SNAs is in a separate well of a multiwell plate.

23. The method of any one of claims 1-22, wherein the SAM comprises an immobilizing moiety that interacts with and immobilizes the substrate and the product.

24. The method of claim 23, wherein the immobilizing moiety comprises a maleimide, a thiol, an alkyne, an azide, an amine, or a carboxyl group.

25. The method of any one of claims 23-24, wherein (i) the immobilizing moiety comprises a maleimide and the substrate and the product each comprise an alkane thiol; (ii) the immobilizing moiety comprises an alkane thiol and the substrate and the product each comprise a maleimide; (iii) the immobilizing moiety comprises an alkyne and the substrate and the product each comprise an azide; (iv) the immobilizing moiety comprises an azide and the substrate and the product each comprise an alkyne; (v) the immobilizing moiety comprises an amine and the substrate and the product each comprise a carboxyl group; or (vi) the immobilizing moiety comprises a carboxyl group and the substrate and the product each comprise an amine, so as to form a chemical bond between the immobilizing moiety and the substrate.

26. The method of any one of claims 1-25, wherein the enzyme is a deacetylase, acetyltransferase, esterase, phosphorylase/kinase, phosphatase, protease, methylase, demethylase, or a DNA or RNA modifying enzyme.

27. The method of claim 26, wherein the phosphatase is secreted embryonic alkaline phosphatase.

28. The method of claim 26, wherein the deacetylase is KDAC8.

29. The method of claim 26, wherein the esterase is cutinase or acetylcholine esterase.

30. The method of claim 26, wherein the protease is TEV.

31. The method of any one of claims 26-30, wherein the substrate comprises an acylated peptide and the product comprises a deacylated peptide.

32. The method of any one of claims 26-30, wherein the substrate comprises a deacylated peptide and the product comprises an acylated peptide.

33. The method of claim 26, wherein the substrate comprises a phosphorylated peptide and the product comprises a dephosphorylated peptide.

34. The method of claim 26, wherein the substrate comprises a dephosphorylated peptide and the product comprises a phosphorylated peptide.

35. The method of claim 26, wherein the substrate comprises a methylated peptide and the product comprises a demethylated peptide.

36. The method of claim 26, wherein the substrate comprises a demethylated peptide and the product comprises a methylated peptide.
